(19) United States
(12) Patent Application Publication
(10) Pub. No.: US 2002/0128675 A1
(43) Pub. Date: Sep. 12, 2002

(54) METHOD AND APPARATUS FOR ENDOSCOPIC DISRUPTION OF LOWER ESOPHAGEAL RING AND STRICTURES

(76) Inventor: CHARLES W. RANDALL, SAN ANTONIO, TX (US)

Correspondence Address: WAYNE J COLTON INC, THE MILAM BUILDING SUITE 1032, 115 EAST TRAVIS STREET, SAN ANTONIO, TX, 78205, US

(21) Appl. No.: 09/430,900

(22) Filed: Nov. 1, 1999

Related U.S. Application Data
(60) Provisional application No. 60/134,435, filed on May 17, 1999.

(51) Int. Cl. A61B 17/32

(52) U.S. Cl. 606/174

(57) ABSTRACT

An improved tool for endoscopic disruption of lower esophageal ring generally comprises scissors adapted to be inserted into a patient's esophagastric junction through a flexible endoscope. The scissors pair comprises a blunt tipped instrument having a pair of short cutting edges operable through an axially disposed cable or the like. In practice, the instrument is inserted into a flexible endoscope and thereby orally inserted into the patient's lower esophagus. Once inserted into the lower esophagus, the instrument is utilized to precisely sever a lower esophageal ring at a plurality of radial locations. Once effectively disrupted, any associated shelf effect will generally self-correct. Other procedures, such as hiatal hernia repair, may then be completed, whereafter the endoscope may be removed from the patient. The procedure should require only about 20 minutes to complete and will result in a minimum of patient discomfort as well as instant relief from the symptoms of LER.

### RELATED APPLICATION

This application claims, under 35 USC §119(e), all available benefit of the filing of U.S. provisional patent application Serial No. 60/134,435 filed May 17, 1999. By this reference, the full disclosure, including the drawings, of U.S. provisional patent application Serial No. 60/134,435 is incorporated herein as though now set forth in its entirety.

### FIELD OF THE INVENTION

The present invention relates to an apparatus for use in endoscopic surgery. More particularly, the invention relates to an endoscopic apparatus specifically adapted for surgical disruption of the lower esophageal, or Schatzki's, ring.

### BACKGROUND OF THE INVENTION

First reported in 1953 by Schatzki and Gary as a ring-like structure at the juncture of the esophageal and gastric mucosa, lower esophageal, or Schatzki's, ring (“LER”) remains controversial as to exact location, etiology and clinical importance. It is generally agreed, however, that if left untreated the incomplete diaphragm in the lower esophageal lumen that characterizes this thin, submucosal, scar can result in serious complications of swallowing. In most cases, LER presents as episodic or progressive dysphagia although, in the extreme, it can progress into a total obstruction of the esophagus. In any case, it is generally accepted standard of care to treat symptomatic LER through surgical interruption or dilation of the ring.

In most cases, a Schatzki's ring is treated by dilation with a large bougie or balloon. While such dilation generally produces immediate relief, recurrence is common and complete cure is almost never observed. As a result, the procedure must be repeated as often as every few years. Although a relatively safe modality, dilation does carry some risks, occasionally including excessive bleeding or perforation of the esophageal wall. Additionally, in some cases the balloon or bougie is not of sufficient diameter to effectively disrupt the LER.

As an alternative to balloon or bougie dilations, therefore, a LER may be surgically interrupted through an open procedure such as laparoscopy or, in extreme cases, gastrotomy via thoracotomy. While probably more effective at preventing recurrence, these open procedures obviously involve dramatically increased trauma to the patient and should therefore be avoided if possible. As a compromise, some surgeons have attempted endoscopic disruption of the LER through electrosurgical radial incision. This, however, is thought to present excessive risk of esophageal perforation and so is not recommended as a general practice.

With the foregoing disadvantages of the prior art in mind, it is an object of the present invention to generally improve over the prior art by providing a treatment modality wherein a lower esophageal ring may be safely and effectively disrupted with minimum patient trauma and reduced likelihood for recurrence.

It is a further object of the present invention to provide such a modality wherein the associated costs are minimized, performance time is reduced and surgical procedures are simplified.

### SUMMARY OF THE INVENTION

In accordance with the foregoing objects, the present invention—an improved tool for endoscopic disruption of lower esophageal ring—generally comprises scissors adapted to be inserted into a patient's esophagastric junction through a flexible endoscope. According to the preferred embodiment of the present invention, the scissors pair comprises a blunt tipped instrument having a pair of short cutting edges, preferably operable through an axially disposed cable.

In practice, the improved instrument of the present invention is inserted into a flexible endoscope and thereby orally inserted into the patient's lower esophagus. Once inserted into the lower esophagus, the instrument is utilized to precisely sever the lower esophageal ring at a plurality of radial locations. Once effectively disrupted, any associated shelf effect will generally self-correct. Other procedures, such as hiatal hernia repair, may then be completed, whereafter the endoscope may be removed from the patient. The procedure of the present invention should require only about 20 minutes to complete and will result in a minimum of patient discomfort as well as instant relief from the symptoms of LER.

Finally, many other features, objects and advantages of the present invention will be apparent to those of ordinary skill in the relevant arts, especially in light of the foregoing discussions and the following drawings, exemplary detailed description and appended claims.

### BRIEF DESCRIPTION OF THE DRAWINGS

Although the scope of the present invention is much broader than any particular embodiment, a detailed description of the preferred embodiment follows together with illustrative figures, wherein like reference numerals refer to like components, and wherein:

FIG. 1 shows, in perspective view, endoscopic scissors as specifically adapted for use in disruption of lower esophageal ring;

FIG. 2 shows, in perspective view, a flexible endosope of an appropriate character for use with the apparatus of FIG. 1;

FIG. 3 shows, in cross-sectional view, a human esophagastric junction with lower esophageal ring present at the juncture of esophageal and gastric mucosa and associated shelf defect present in the subhiatal ring; and

FIG. 4 shows, in cross-sectional view, the human esophagastric junction of FIG. 3 after endoscopic disruption of the lower esophageal ring according to the preferred method of the present invention and natural correction thereafter of the shelf defect.

### DETAILED DESCRIPTION OF THE PREFERRED EMBODIMENT

Although those of ordinary skill in the art will readily recognize many alternative embodiments, especially in light of the illustrations provided herein, this detailed description is exemplary of the preferred embodiment of the present invention, the scope of which is limited only by the claims appended hereto.

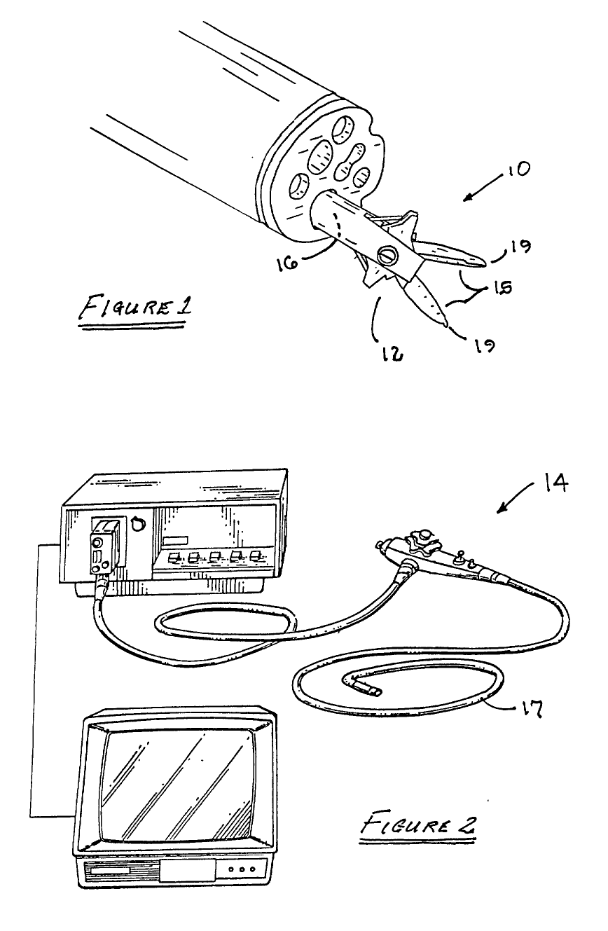

Referring now to the Figures, an improved tool 10 for endoscopic disruption of lower esophageal ring 11 is described in detail. As shown particularly in FIG. 1, the improved tool 10 of the present invention generally comprises scissors 12 adapted to be inserted into a patient's esophagastric junction 13 through a flexible endoscope 14. According to the preferred embodiment of the present invention, the scissors pair 12 comprises a blunt tipped instrument having a pair of short cutting edges 15. The cutting edges 15 are preferably operable through an axially disposed cable 16, as has been employed by those of ordinary skill in the art for instruments of similar character but for different use. By way of example only, one such instrument is detailed in U.S. Pat. No. 4,714,075 issued Dec. 22, 1987 for a BIOPSY CHANNEL FOR ENDOSCOPE. By this reference, the full disclosure of U.S. Pat. No. 4,714,075 is incorporated herein as though now set forth in its entirety.

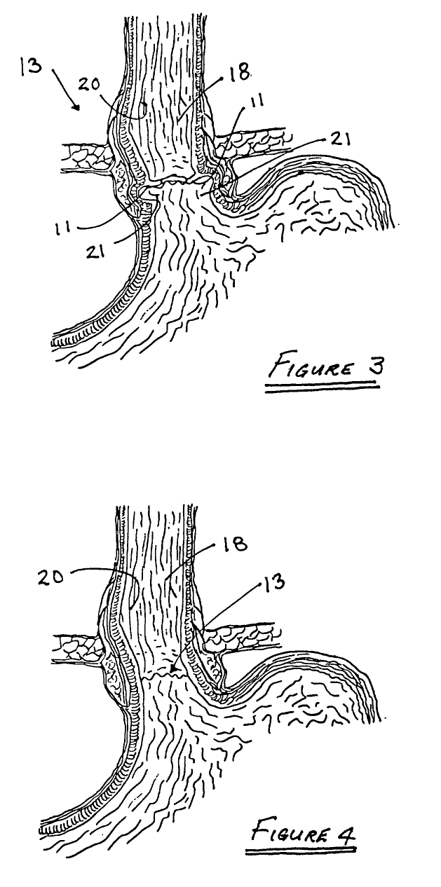

In practice, the improved instrument 10 of the present invention is inserted into a flexible endoscope 14 as depicted in FIG. 2. As known by those of ordinary skill in the art, such a flexible endoscope 14 allows the surgeon to insert any of a variety of instruments, as well as optical and lavage capabilities, into a patient cavity through a natural or surgically created orifice. In the present case, the flexible portion 17 of the endoscope 14 is orally inserted into the patient's lower esophagus 18, depicted in FIGS. 3 and 4. Although those of ordinary skill in the art will recognize that many substantially equivalent alternatives to the device as shown in FIG. 1 may be implemented, it is critical to the present invention that the resulting instrument be extremely flexible for unimpeded maneuvering into the required surgical position. Likewise, it is preferred that the instrument be of sufficiently low profile as to not obstruct the surgeon's view through the endoscope 14. Finally, blunt tips 19 are preferred in order to prevent inadvertent perforation of the esophageal wall 20.

Once inserted into the lower esophagus 18, the instrument 10 of the present invention is utilized to precisely sever the ring 11 at a plurality of radial locations. Once effectively disrupted, any associated shelf effect 21 will generally self-correct, as depicted in FIGS. 3 and 4. Other procedures, such as hiatal hernia repair, may then be completed, whereafter the endoscope 14 may be removed from the patient. The procedure of the present invention should require only about 20 minutes and will result in a minimum of patient discomfort as well as instant relief from the symptoms of LER.

Although other existing instruments, such as the biopsy forceps depicted in U.S. Pat. No. 4,714,075 may be utilized to disrupt a LER 11, it is preferred that the scissors arrangement of the present invention be employed in order to facilitate surgical control and reduce local trauma. Additionally, biopsy forceps and other instruments of like character are generally ineffective without at least some prior ballooning to identify weak portions of the LER 11. The instrument 10 of the present invention, on the other hand, may be used to quickly and effectively disrupt the LER 11 at a plurality of locations without regard for relative ring thickness and with maximum surgical control and minimum local trauma. As opposed to the more risky electrosurgical radial incision, the method of the present invention requires little or no special training and may be performed in approximately half the time required for ballooning and tearing with biopsy forceps. The latter benefit, of course, results in less anesthesia time for the patient, reducing the costs associated with the procedure as well as risk to the patient.

While the foregoing description is exemplary of the preferred embodiment of the present invention, those of ordinary skill in the relevant arts will recognize the many variations, alterations, modifications, substitutions and the like as are readily possible, especially in light of this description, the accompanying drawings and claims drawn thereto. For example, the instrument 10 of the present invention is also well-adapted for the similar endoscopic treatment of strictures such as may result from pyloric stenosis—a narrowing of the gastric pyloris due to peptic ulcer—and other like maladies of the duodenum. In any case, because the scope of the present invention is much broader than any particular embodiment, the foregoing detailed description should not be construed as a limitation of the scope of the present invention, which is limited only by the claims appended hereto.

What is claimed is:

1. A cutting instrument for endoscopic disruption of lower esophageal ring, said cutting instrument comprising:
an elongate, flexible insertion member, said insertion member having a first, proximal end and a second, distal end, said first, proximal end being adapted to interface with an endoscopic controller;
a first and a second cutting edge dependently secured to said insertion member at said second, distal end, said cutting edges being adapted to traverse a flexible endoscopic channel; and
an actuator associated with said insertion member, said actuator being adapted to operate said cutting edges according to the endoscopic controller.

2. The cutting instrument as recited in claim 1, wherein said first cutting edge is hingedly attached to said second cutting edge.

3. The cutting instrument as recited in claim 2, wherein said cutting edges each comprise a blunt tip adapted to perturb a fleshy tissue substantially without injury thereto.

4. The cutting instrument as recited in claim 2, wherein said cutting edges comprise a profile substantially less than that of a conventional endoscopic channel such that said cutting edges may be operated without significant impediment of an operator's view through the endoscopic channel.

5. The cutting instrument as recited in claim 2, wherein said flexible insertion member is adapted to reverse the orientation of said cutting edges substantially within a circle having a radius less than about twice the length of said cutting edges.

6. A cutting instrument for endoscopic severance of animal tissues, said cutting instrument comprising:
an elongate, flexible insertion member, said insertion member having a first, proximal end and a second, distal end, said first, proximal end being adapted to interface with an endoscopic controller;
scissors dependently secured to said insertion member at said second, distal end, said scissors being adapted to traverse a flexible endoscopic channel; and
an actuator associated with said scissors and said insertion member, said actuator being adapted to operate said scissors and direct said insertion member according to the endoscopic controller.

7. The cutting instrument as recited in claim 6, wherein said actuator is adapted to flex said second, distal end of said insertion member.

8. The cutting instrument as recited in claim 7, wherein said actuator is further adapted to cause a reversal of the orientation of said scissors relative to the endoscopic channel.

9. The cutting instrument as recited in claim 8, wherein the reversal in orientation of said scissors may be accomplished substantially within a circle having a radius less than about twice the length of said scissors.

10. The cutting instrument as recited in claim 9, wherein said scissors comprise a pair of bluntly tipped cutting edges.

11. The cutting instrument as recited in claim 10, wherein the blunt tip of each said cutting edge is adapted to displace an animal tissue substantially without cutting thereof.

12. A method for endoscopic disruption of a gastrointestinal stricture, said method comprising the steps of:
inserting a pair of cutting edges through a highly flexible endoscopic channel;
maneuvering said cutting edges into the proximity of the gastrointestinal stricture; and
severing the gastrointestinal stricture with said cutting edges.

13. The method as recited in claim 12, wherein said cutting edges comprise scissors.

14. The method as recited in claim 13, wherein each said cutting edge comprises a substantially blunt tip.
